(19) United States
(12) Patent Application Publication
(10) Pub. No.: US 2016/0310628 A1
(43) Pub. Date: Oct. 27, 2016

(54) Ion-generating Apparatus and Method for Sterilization and for Removing Smog

(71) Applicant: LUFTMED GMBH, Muenchen (DE)

(72) Inventors: Fang JIANG, Guangdong (CN); Tieming YAO, Guangdong (CN); Qingjie ZENG, Guangdong (CN); Jinhua TANG, Guangdong (CN); Yinhua TANG, Guangdong (CN); Hui WANG, Guangdong (CN)

(73) Assignee: LUFTMED GMBH, Muenchen (DE)

(21) Appl. No.: 15/104,084

(22) PCT Filed: Dec. 16, 2014
(86) PCT No.: PCT/EP2014/078101

§ 371 Date: Jun. 13, 2016

(30) Foreign Application Priority Data
Dec. 17, 2013 (CN) 201310698271.4
Dec. 17, 2013 (CN) 201310698699.9

(51) Int. Cl. A61L 9/22; B03C 3/82; B03C 3/68; F24F 3/16

(52) U.S. Cl. CPC A61L 9/22; F24F 3/166; B03C 3/82; B03C 3/68; F24F 2003/1682; F24F 2011/0023

(57) ABSTRACT

The present invention discloses an ion-generating apparatus for sterilization and removing smog, comprising a housing consisted of a shielding panel and a casing, an ion generator configured within the housing for generating negative ions O₂⁻(H₂O)X and positive ions H⁺(H₂O)Y, wherein X and Y are any natural numbers. The ion generator comprises a sterilizing module and a smog-removing module, wherein both modules are dominated by one controller, to generate positive and negative ions periodically. The present invention combines the sterilizing module and the smog-removing module together to implement two functions, i.e. periodically sterilization and smog-removal, and to a method for sterilization and for removing smog employing the ion generator.

The present invention relates to an ion-generating apparatus containing an ion generator for sterilization and for removing smog and to a method for sterilization and for removing smog employing the ion generator.

Air pollutants in the indoor living environment or working environment mainly comprise total volatile organic compounds (TVOC), smog, tobacco smoke, as well as bacteria, fungi (e.g. mold). People who have allergies often are sensitive against dust and certain types of pollen and would therefore also consider dust and pollen being air pollutant. Examples of pollutants of mostly chemical nature are those which are often found in the living environment after renovation and include formaldehyde, benzene and other volatile organic compounds. All these air pollutants can negatively affect the health of a human or animal. In certain regions, a large amount of dust and suspended particulates is found in the outdoor atmospheric environment which often results in an indoor pollution. However, since the indoor environment is usually a more or less confined space with often stagnant air having a low level of oxygen, and if smokers are present, also with unhealthy air, this indoor environment could also have adverse effects on the health of a human and animal.

Methods for air purifying as well as air purifying apparatus using different kind of filters are known. Nowadays, such filter air purification devices are also combined with an air-ionization technique. One problem which occur with the known air-ionization devices for air purification is, that such devices often also emit unhealthy ions such as ozone. Additionally to that, the air purification devices presently on the market consume a lot of energy as they are often just operate in the “on-” or “off-mode”.

Therefore, there is a need for efficiently and reliably purifying the indoor environment from any sort of pollution, such as TVOC or particulate matter (PM), found in smog, and biological material such as bacteria, virus and pollen. It is moreover desirable to have an apparatus and method for air purification which is highly effective and which, at the same time, is energy saving. It is costly and impractical to activate the ion generator for long time due to energy consumption.

It would be desirable to provide an ion-generating apparatus for sterilization and removing smog to alleviate the above-described problems. The apparatus can facilitate cleaning of indoor air with energy-saving. Moreover, it would be desirable to provide a method for sterilization and removing smog particles periodically according to the changes in the quantity of particles in ambient air to alleviate the above-described problems. The problem is solved by the provision of an ion-generating apparatus containing the inventive ion generator and the inventive method for removing smog and for sterilization by using the ion generator as described and as claimed.

The ion generator according to this invention is advantageous as it can operate in a sterilization mode (sterilization function) and in a smog-removing mode (smog-removing function). It also contains just one controller which controls whether the ion generator operates in the sterilizing or in the smog-removing mode so that the desired type of positive and negative ions are periodically generated. This set-up is energy saving and thus environmental friendly.

With regard to the above functions, the invention is directed to a ion-generating apparatus for sterilization and for removing smog which apparatus comprises a housing consisting of a shielding panel and a casing; and an ion generator for generating negative ions O₂⁺(H₂O)x and positive ions H⁺(H₂O)Y, wherein x and y are any natural numbers and whereas the ion generator is mounted within the housing.

In the sterilizing mode, the ion generator employs the sterilizing module which contains a sterilization circuit consisting of a first transformation unit outputting 100 V to 120 V voltage and a second transformation unit outputting voltage in the range of 3.5 kV to 4 kV, a positive discharge needle and a negative discharge needle. The first transformation unit, the second transformation unit and the controller of the ion generator are connected in turn.

The first transformation unit comprises a first transformer, a first diode and a first resistor connected in turn to the first end of the primary coil of the first transformer, a first triode connected to the second end of the primary coil of the first transformer, and a second resistor and a third resistor serial-connected one another then parallel-connected to the secondary coil of the first transformer. The positive electrode of the first diode is connected to the power source and the negative electrode of the first diode is connected to the first resistor. The emitter of the first triode is grounded and the base of the first triode is connected to the controller. The connection end of the second resistor and the third resistor is connected to the controller.

The second transformation unit comprises a second transformer, a third diode and a second diode connected in serial then connected to the first end of the primary coil of the second transformer, a sixth diode and a seventh diode connected to the second end of the secondary coil of the second transformer, the connection end of the third diode and the second diode is grounded through a first capacitor, the second end of the primary coil of the second transformer is also grounded. The negative electrode of the sixth diode is connected to the positive discharge needle and the positive electrode of the sixth diode is connected to the second end of the secondary coil of the second transformer. The positive electrode of the seventh diode is connected to the negative discharge needle and the negative electrode the seventh diode is connected to the second end of the secondary coil of the second transformer.

In the smog-removing mode, the ion generator employs the smog-removing module which comprises a smog-removing circuit and brush-shaped ion discharger. This circuit comprises a third transformer, a fourth diode and a sixth resistor connected to the first end of the primary coil of the third transformer, a second triode connected to the second end of the primary coil of the third transformer, a fifth diode and a seventh resistor serial-connected in turn then configured between the first end of the secondary coil of the third transformer and the brush-shaped ion discharger, and a fourth resistor and a fifth resistor serial-connected one another then parallel-connected to the secondary coil of the third transformer. The positive electrode of the fourth diode is connected to the power source. The emitter of the second triode is grounded and the base of the second triode is connected to the controller. The connection end of the fourth resistor and the fifth resistor is connected to the controller. The positive electrode of the fifth diode is connected to the seventh resistor and the negative electrode of the fifth diode is connected to the first end of the secondary coil of the third transformer, the output voltage of the secondary coil of the third transformer is in the range of 4.5 kV to 6 kV. Having a smog-removing function is advantageous when smog is influenced by a haze.

In the sterilization mode, the controller outputs five square waves with a duty ratio of 0.5 and a cycle of 0.066 milliseconds to the sterilizing module every two milliseconds.

In the smog-removing mode, the controller outputs a square wave with a duty ratio of 0.5 and a cycle of 6 milliseconds to the smog-removing module.

The shielding panel comprises a first hole corresponding to the positive hydrogen ion discharge needle, a second hole corresponding to the negative oxygen ion discharge needle, and negative ions exhausting holes corresponding to the brush-shaped ion dischargers respectively. There are two brush-shaped ion dischargers symmetrically provided on the two sides of a printed circuit board (PCB) which is configured with the sterilization circuit and the smog-removing circuit. The method for sterilization and for removing smog particles employs the ion generator according to the invention and comprises the following steps: detecting the quantity of the particles in ambient air; determining whether the quantity of the particles exceed the limits; if yes, the ion generator produces negative and positive ions in the sterilization mode; otherwise, the ion generator periodically makes corona discharge which ionizes the air to form negative oxygen ions in the smog-removing mode; then repeat the above steps.

When operating in the sterilization mode, the ion generator produces negative and positive ions with the following steps: every two milliseconds a controller outputs five sterilizing square waves with a duty ratio of 0.5 and a cycle of 0.066 milliseconds to the sterilizing module containing the sterilizing circuit, the positive and the negative discharge needle whereas the sterilizing circuit comprises a first transformation unit outputting Voltage in the range of 100 V to 120 V and a second transformation unit outputting voltage in the range of 3.5 kV to 4 kV. The controller, the first transformation unit and the second transformation unit are connected in turn. When the sterilizing module receives the sterilizing square waves it produces an output voltage in the range of 3.5 kV to 4 kV and positive and negative ions are released through the positive and the negative discharge needle respectively.

Further, the ion generator makes corona discharge periodically with the following steps: the controller outputs a smog-removing square wave with a duty ratio of 0.5 and a cycle of 6 milliseconds to a smog-removing module; the smog-removing circuit comprises a third transformer, a fourth diode and a sixth resistor connected to the first end of the primary coil of the third transformer, a second triode connected to the second end of the primary coil of the third transformer, a fifth diode and a seventh resistor serial-connected in turn then configured between the first end of the secondary coil of the third transformer and brush-shaped ion dischargers, and a fourth resistor and a fifth resistor serial-connected one another then parallel-connected to the secondary coil of the third transformer. The positive electrode of the fourth diode is connected to the power source, the emitter of the second triode is grounded and the base of the second triode is connected to the controller, the connection end of the fourth resistor and the fifth resistor is connected to the controller, the positive electrode of the fifth diode is connected to the seventh resistor and the negative electrode of the fifth diode is connected to the first end of the secondary coil of the third transformer, the output voltage of the secondary coil of the transformer is 4.5 kV to 6 kV. The smog-removing module receives the smog-removing square wave, produces 4.5 kV to 6 kV output voltage and periodically makes corona discharge through the brush-shaped ion dischargers.

The present invention will be described in details below with reference to the accompanying figures. The exemplary embodiments are only used for description and explanation of the present invention, without limiting the invention thereto.

The present invention relates to an ion-generating apparatus for sterilization and for removing smog, which is able to remove smog, e.g. the particles of air pollutants, or sterilize the bacteria or other pollutant of biological origin.

Under the sterilization mode, the apparatus generates negative ions O₂⁻(H₂O)X and positive ions H⁺(H₂O)Y, wherein x and y are any natural numbers. These ions are exhaled to the air, resulting in the oxidation reaction to form hydrogen peroxide H₂O₂ or a free radical •OH, both serving as active species which are able to destroy floating bacteria in the air and thus to sterilize the air.

Under the smog-removing mode, the two brush-shaped ion dischargers operate simultaneously make corona discharge periodically according to the instructions. Large numbers of free electrons are omitted and thus ionizing the air forming negative oxygen ions. The omitted electrons will be mostly captured by oxygen molecules as oxygen has a very high electron affinity compared to other gases found in air. These negative oxygen ions are exhausted as high negative pressure. Via action of electric field as well as through the wind of a fan contained in the apparatus the ions spread in the air removing the pollutant (e.g. PM contained in smog, dust and pollen) and thus purifying the air to yield “fresh air”. This mode of action is not suitable for removing odor.

In an [embodiment 1] the invention refers to an ion-generating apparatus for sterilization and removing smog, comprising a housing consisted of a shielding panel and a casing, and an ion generator configured within the housing for generating negative ions O₂⁻(H₂O)X and positive ions H⁺(H₂O), wherein x and y are any natural numbers, wherein the ion generator comprises a sterilizing module and a smog-removing module, both modules are dominated by one controller to generate positive and negative ions periodically. In an [embodiment 2] the invention is directed to an ion-generating apparatus according to [embodiment 1], wherein the sterilizing module comprises a sterilizing circuit consisting of a first transformation unit outputting 100 V-120 V voltage and a second transformation unit outputting 3.5 kV-4 kV voltage, a positive discharge needle and a negative discharge needle, wherein the controller, the first transformation unit and the second transformation unit are connected in turn.

In an [embodiment 3] the invention is directed to an ion-generating apparatus according to [embodiment 2], wherein the first transformation unit further comprises a first transformer, a first diode and a first resistor connected in turn then connected to the first end of the primary coil of the first transformer, a first triode connected to the second end of the primary coil of the first transformer, and a second resistor and a third resistor connected in series one another then parallel-connected to the secondary coil of the first transformer, wherein the positive electrode of the first diode is connected to the power source and the negative electrode of the first diode is connected to the first resistor, the emitter of the first triode is grounded and the base of the first triode is connected to the controller, and the connection end of the second resistor and the third resistor is connected to the controller.

In an [embodiment 4] the invention is directed to an ion-generating apparatus according to [embodiment 3], wherein the second transformation unit further comprises: a second transformer, a third diode and a second diode connected in series then connected to the first end of the primary coil of the second transformer, and a sixth diode and a seventh diode connected to the second end of the secondary coil of the second transformer, wherein the connection end of the third diode and the second diode is grounded through a first capacitor, the second end of the primary coil of the second transformer is grounded, the negative electrode of the sixth diode is connected to the positive discharge needle and the positive electrode of the sixth diode is connected to the second end of the secondary coil of the second transformer, and the positive electrode of the seventh diode is connected to the negative discharge needle and the negative electrode of the seventh diode is connected to the second end of the secondary coil of the second transformer.

In an [embodiment 5] the invention is directed to an ion-generating apparatus according to [embodiment 2], wherein the smog-removing module comprises a smog-removing circuit and brush-shaped ion dischargers, wherein the smog-removing circuit further comprises a third transformer, a fourth diode and a sixth resistor connected to the first end of the primary coil of the third transformer, a second triode connected to the second end of the primary coil of the third transformer, a fifth diode and a seventh resistor connected in series in turn and configured between the first end of the secondary coil of the third transformer and the brush-shaped ion dischargers, and a fourth resistor and a fifth resistor connected in series one another then parallel-connected with the secondary coil of the third transformer; wherein the positive electrode of the fourth diode is connected to the power source, the emitter of the second triode is grounded and the base of the second triode is connected to the controller, the connection end of the fourth resistor and the fifth resistor is connected to the controller, the positive electrode of the fifth diode is connected to the seventh resistor and the negative electrode of the fifth diode is connected to the first end of the secondary coil of the third transformer, the output voltage of the secondary coil of the third transformer is 4.5 kV-6 kV.

In an [embodiment 6] the invention is directed to an ion-generating apparatus according to [embodiment 1] wherein the controller outputs five square waves with a duty cycle of 0.5 and a cycle of 0.066 milliseconds to the sterilizing module every two milliseconds.

In an [embodiment 7] the invention is directed to an ion-generating apparatus according to [embodiment 1] wherein the controller outputs a square wave with a duty cycle of 0.5 and a cycle of 6 milliseconds to the smog-removing module.

In an [embodiment 8] the invention is directed to an ion-generating apparatus according to [embodiment 5], wherein the shielding panel comprises a first hole corresponding to the positive hydrogen ion discharge needle, a second hole corresponding to the negative oxygen ion discharge needle, and negative ions exhausting holes corresponding to the brush-shaped ion dischargers; wherein there are two brush-shaped ion dischargers symmetrically provided inside the housing.

The method for sterilization and removing smog particles according to the present invention, comprising the steps S10 to S20 as following and as given in FIG. 6:

Step S10: detecting the quantity of the particles in ambient air. The quantity of the particles in ambient air may be detected by the detecting module in the ion apparatus. An air extractor can be used for extracting ambient air into the apparatus such that the quantity of the particles in ambient air can be detected precisely.

Step S20: determining whether the quantity of the particles exceed the limit. Comparing the detected quantity of the particles with the predetermined quantity of the particles so as to determine whether the detected quantity of the particles in ambient air is exceeded the limit. If the detected quantity of the particles exceeds the predetermined quantity of particles, then performing steps S201 and S202. Otherwise, performing steps S210 and S211. Repeat Step S10 after finishing Step S202 or Step S211.

Steps S201, S202, S210 and S211 are as follows:

Step S201: the controller outputs a smog-removing square wave with a duty ratio of 0.5 and a cycle of 6 milliseconds to the smog-removing module.

Step S202: the smog-removing module receives the smog-removing square wave, produces 4.5 kV-6 kV output voltage and makes corona discharge periodically through the brush-shaped ion dischargers.

Step S210: the controller outputs five sterilizing square waves with a duty ratio of 0.5 and a cycle of 0.066 milliseconds to the sterilizing module every two milliseconds.

Step S211: the sterilizing module receives the sterilizing square waves, produces 3.5 kV-4 kV output voltage and releases positive and negative ions through the positive and negative discharge needles respectively.

In an [embodiment A] the invention is directed to a method for sterilization and for removing smog particles using an ion generator comprising the following steps: detecting the quantity of particles in ambient air; determining whether the quantity of particles exceed the limits; if limit is exceeded, then an ion generator produces negative and positive ions; if limit is not exceeded, then the ion generator periodically makes corona discharges which ionizes air to form negative oxygen ions; repeating before steps.

In an [embodiment B] the invention is directed to the method according to [embodiment A] employing an ion generator comprising a controller and a sterilizing module, which produces negative and positive ions and which method further comprises the following steps for generating positive or negative ions:
if limit is exceeded, then the ion generator produces negative and positive ions by employing a controller which outputs five sterilizing square waves with a duty ratio of 0.5 and a cycle of 0.066 milliseconds to a sterilizing module every two milliseconds whereas the sterilizing module comprises a sterilizing circuit, a positive discharge needle and a negative discharge needle, and whereas the sterilizing circuit comprises a first transformation unit outputting voltage in the range of 100 V-120 V and a second transformation unit outputting voltage in the range of 3.5 kV to 4 kV, whereas the controller, the first transformation unit and the second transformation unit are connected in turn, and whereas the sterilizing module which receives the sterilizing square waves produces voltage in the range of 3.5 kV to 4 kV releases positive and negative ions through positive and negative discharge needles respectively.

In an [embodiment C] the invention is directed to the method according to [embodiment B] whereas the first transformation unit comprises:
a first transformer, a first diode and a first resistor connected in turn to the first end of the primary coil of the first transformer, a first triode connected to the second end of the primary coil of the first transformer, and a second resistor and a third resistor serial-connected one another then parallel-connected to the secondary coil of the first transformer, the positive electrode of the first diode is connected to the input power and the negative electrode of the first diode is connected to the first resistor, the emitter of the first triode is grounded and the base of the first triode is connected to the controller, and the connection end of the second resistor and the third resistor is connected to the controller.

In an [embodiment D] the invention is directed to the method according to [embodiment C], wherein the second transformation unit comprises: a second transformer, a third diode and a second diode connected in serial then connected to the first end of the primary coil of the second transformer, a sixth diode and a seventh diode connected to the second end of the secondary coil of the second transformer, the connection end of the third diode and the second diode is grounded through a first capacitor, the second end of the primary coil of the second transformer is grounded, the negative electrode of the sixth diode is connected to the positive discharge needle and the positive electrode of the sixth diode is connected to the second end of the secondary coil of the second transformer, and the positive electrode of the seventh diode is connected to the negative discharge needle and the negative electrode of the seventh diode is connected to the second end of the secondary coil of the second transformer.

In an [embodiment E] the invention is directed to the method according to [embodiment A], wherein the ion generator periodically makes corona discharge further comprising the following step:
the controller outputs a smog-removing square wave with a duty ratio of 0.5 and a cycle of 6 milliseconds to a smog-removing module, whereas the smog-removing circuit comprises a third transformer, a fourth diode and a sixth resistor connected to the first end of the primary coil of the third transformer, a second triode connected to the second end of the primary coil of the third transformer, a fifth diode and a seventh resistor serial-connected in turn and configured between the first end of the secondary coil of the third transformer and brush-shaped ion dischargers, and a fourth resistor and a fifth resistor serial-connected one another then parallel-connected to the secondary coil of the third transformer; wherein the positive electrode of the fourth diode is connected to the input power, the emitter of the second triode is grounded and the base of the second triode is connected to the controller, the connection end of the fourth resistor and the fifth resistor is connected to the controller, the positive electrode of the fifth diode is connected to the seventh resistor and the negative electrode of the fifth diode is connected to the first end of the secondary coil of the third transformer, the output voltage of the secondary coil of the transformer is in the range of 4.5 kV to 6 kV, and wherein the smog-removing module receives the smog-removing square wave, produces an output voltage in the range of 4.5 kV to 6 kV and periodically makes corona discharge through the brush-shaped ion dischargers.

The method is embodied by the ion generator as described herein.

FIG. 1 is a structure schematic diagram of the ion-generating apparatus for sterilization and removing smog according to the present invention comprising a shielding panel (1), a casing (2), a printed circuit board (3), brush shaped ion dischargers (4A, 4B), a first hole (7A) and a second hole (7B), negative ions exhausting holes (8A, 8B), a positive discharge needle and a negative discharge needle.

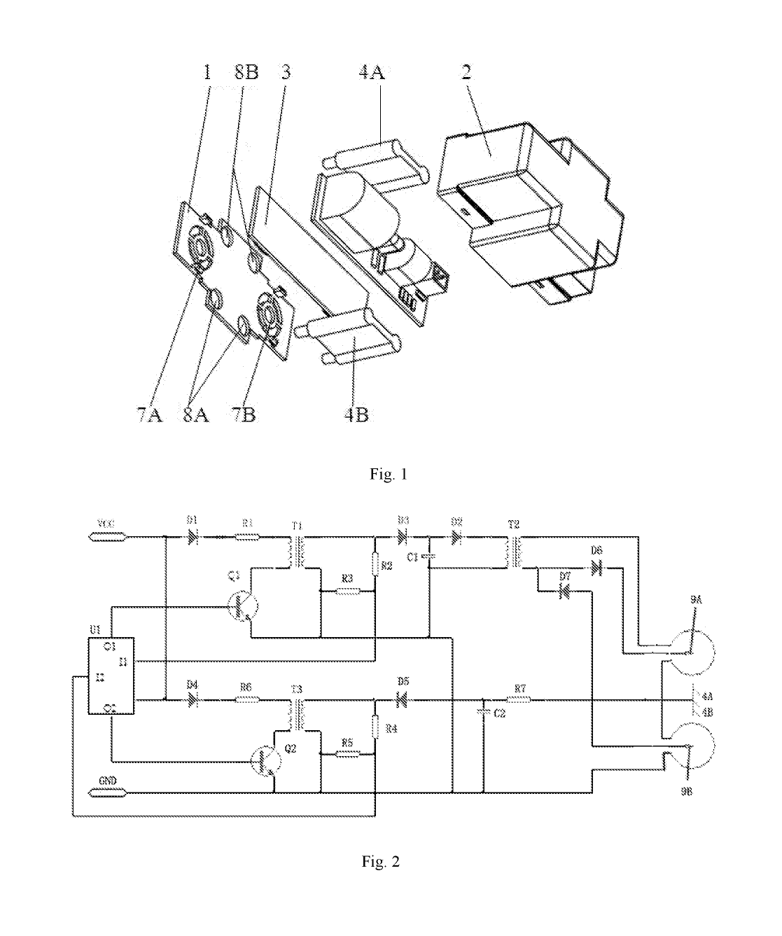

The ion-generating apparatus of this embodiment given in FIG. 1 comprises a housing (not shown in FIG. 1), which housing comprises a shielding panel (1) and a casing (2). An ion generator is provided in the housing. The shielding panel (1) comprises a first hole (7A) which correspond to the positive hydrogen ion discharge needle, a second hole (7B) which correspond to the negative oxygen ion discharge needle, and negative ions exhausting holes (8A) and (8B) which correspond to brush-shaped ion dischargers (4A) and (4B), respectively. The positive hydrogen ion discharge needle and negative oxygen ion discharge needle are placed on the printed circuit board (PCB) (3). The positive (hydrogen) ion discharge needle (9A) and negative (oxygen) ion discharge needle (9B) are only shown in FIG. 2). The negative ions exhausting holes (8A) and (8B) are provided on the shielding panel (1) in a symmetrical way. Such symmetrical structure is beneficial for improving diffusion of the ions and, therefore, for increasing the quantity of ions found in the air.

FIG. 2 is a schematic circuit diagram of the ion-generating apparatus according to the present invention comprising comprises a sterilizing module and a smog-removing module, wherein both modules are dominated by one controller. The controller controls the sterilizing module and the smog-removing module to discharge periodically thus generating positive and negative ions.

The sterilizing module includes a sterilizing circuit, a positive discharge needle (9A) and a negative discharge needle (9B). The sterilizing circuit comprises a first transformation unit outputting 100 V-120 V voltage and a second transformation unit outputting 3.5 kV to 4 kV voltage, wherein the controller U1, the first transformation unit and the second transformation unit are connected in turn.

The first transformation unit comprises a first transformer T1, a first diode D1, a first resistor R1, a first triode Q1, a second resistor R2 and a third resistor R3. The positive electrode of the first diode D1 is connected to the power VCC and the negative electrode of the first diode D1 is connected to the first resistor R1. The other end of the first resistor R1 is connected to the first end of the primary coil of the first transformer T1. The collector of the first triode Q1 is connected to the second end of the primary coil of the first transformer, the base of the first triode Q1 is connected to the end O1 of the controller U1, and the emitter of the first triode Q1 is grounded. The second resistor R2 and the third resistor R3 are connected in serial and then parallel-connected with the secondary coil of the first transformer T1. The connecting end of the second resistor R2 and the third resistor R3 is connected to the end I1 of the controller U1. The first transformer T1 can output 100 V-120 V voltage which supplies to the second diode D2. The second diode D2 will not work if below the required voltage range, and it will break down if over the voltage range.

The second transformation unit comprises a second transformer T2, a second diode D2, a third diode D3, a first capacitor C1, a sixth diode D6 and a seventh diode D7. The third diode D3 and the second diode D2 are connected in serial. The negative electrode of the second diode D2 is connected to the first end of the primary coil of the second transformer T2, the positive electrode of the third diode D3 is connected to the first end of the secondary coil of the first transformer T1. The positive electrode of the sixth diode D6 is connected to the second end of the secondary coil of the second transformer T2 and the negative electrode of the sixth diode D6 is connected to the positive discharge needle 9A. The positive electrode of the seventh diode D7 is connected to the negative discharge needle 9B and the negative electrode of the seventh diode D7 is connected to the second end of the secondary coil of the second transformer T2. The connecting end of the third diode D3 and the second diode D2 is grounded through the first capacitor. The second end of the primary coil of the second transformer T1 is grounded. The second transformation unit outputs 3.5 kV to 4 kV voltage. This voltage must be adapted to the requirement for the ion discharge, however, it must be noted that ozone will be produced in an undesired quantity if the voltage exceeds 4 kV, and if the voltage is below 3.5 kV the quantity of ions is significantly reduced. Operating the ion-generating apparatus at a voltage in the range from 3.5 kV until 4 kV leads to the formation of the highest quantity of the desired ions.

Under the sterilization mode, the end O1 of the controller U1 outputs pulse voltage allowing the triode Q1 on and off continuously so that discontinuous current passes through the primary coil of the first transformer T1. The current is coupled to the secondary coil of the first transformer T1 generating 100 V-120 V alternating current (AC) voltage. This AC voltage is divided by the second resistor R2 and the third resistor R3, and the divided voltage is input to the end I1 of the controller U1. According to this divided voltage, the end O1 of the controller U1 can output the relevant signal to control the conduction time of the first triode Q1 so as to obtain the desired voltage waveform at the secondary coil of the first transformer T1. Then, this voltage is input to the primary coil of the second transformer T2, and thereby 3.5 kV-4 kV AC voltage will be generated at the secondary coil of the second transformer T2. The positive voltage can be discharged by the discharge needle (9A) after the rectification of the rectifying diode D6 so that the hydrogen atoms in the nearby air loss electrons then become positive ions. The negative voltage can be discharged by the discharge needle (9B) after the rectification of the rectifying diode D7 so that the oxygen atoms in the nearby air gain electrons then become negative ions.

The smog-removing module comprises a smog-removing circuit and brush-shaped ion dischargers (4A) and (4B). The smog-removing circuit comprises a third transformer T3, a fourth diode D4, a sixth resistor R6, a fifth diode D5, a fourth resistor R4, a fifth resistor R5, a second triode D2 and a seventh resistor R7. The positive electrode of the fourth diode D4 is connected to the controller U1 and the negative electrode of the fourth diode D4 is connected to the sixth resistor R6. The base of the second triode Q2 is connected to the controller U1, the collector of the second triode Q2 is connected to the second end of the primary coil of the third transformer T3, and the emitter of the second triode Q2 is grounded. The fourth resistor R4 and the fifth resistor R5 are connected in series and then they are parallel-connected with the secondary coil of the third transformer T3, wherein the connecting end of the fourth resistor R4 and the fifth resistor R5 is connected to the end I2 of the controller U1. The negative electrode of the fifth diode D5 is connected to the first end of the secondary coil of the third transformer T3 and the positive electrode of the fifth diode D5 connected to the seventh resistor R7, and the other end of the seventh resistor R7 is connected to the brush-shaped ion dischargers 4A and 4B. The secondary coil of the third transformer T3 outputs 4.5 kV to 6 kV AC voltage. Under the smog-removing mode, the end O2 of the controller U1 outputs pulse voltage allowing the second triode Q2 on and off continuously so that discontinuous current passes through the primary coil of the third transformer T3. The current is coupled to the secondary coil of the third transformer T3 generating 4.5 kV-6 kV AC voltage. This AC voltage is divided by the fourth resistor R4 and the fifth resistor R5, and the divided voltage is inputted to the end I2 of the controller U1. According to this divided voltage, the end O2 of the controller U1 can output the relevant signal to control the conduction time of the second triode Q2 so as to achieve the purpose of controlling output target voltage of the secondary coil of the third transformer T3. The voltage generated by the third transformer T3 is changed to negative voltage after the rectification of the rectifying diode D5, then this negative voltage goes through the seventh resistor R7 and begins to be discharged by the brush-shaped ion dischargers so that the oxygen atoms in the nearby air gain electrons and become negative ions.

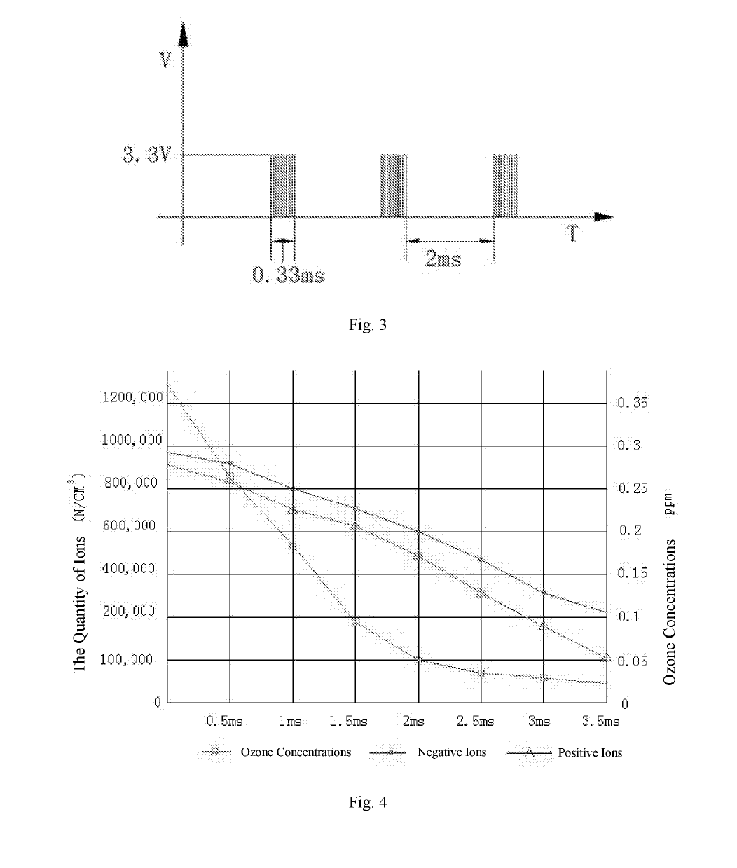

FIG. 3 is a control waveform from the controller of the ion-generating apparatus according to the present invention to the first triode.

The output waveforms of the controller U1 makes the first triode Q1 on and off. The controller U1 in FIG. 3 outputs five square waves with a duty ratio of 0.5 and a cycle of 0.066 milliseconds every two milliseconds. Such a two milliseconds interval is set up for generating more negative ions with less ozone. If the time interval is longer than two milliseconds, the quantity of positive and negative ions is reduced. The reduction increases as the time increases. On the other hand, if the time interval is too short, an undesirable high amount of ozone is produced. Therefore, 2 milliseconds is the optimum time interval and which is demonstrated in the experimental data shown in FIG. 4.

FIG. 4 is an experimental data diagram showing the densities of ozone, negative ions and positive ions caused by the control waveform in FIG. 3.

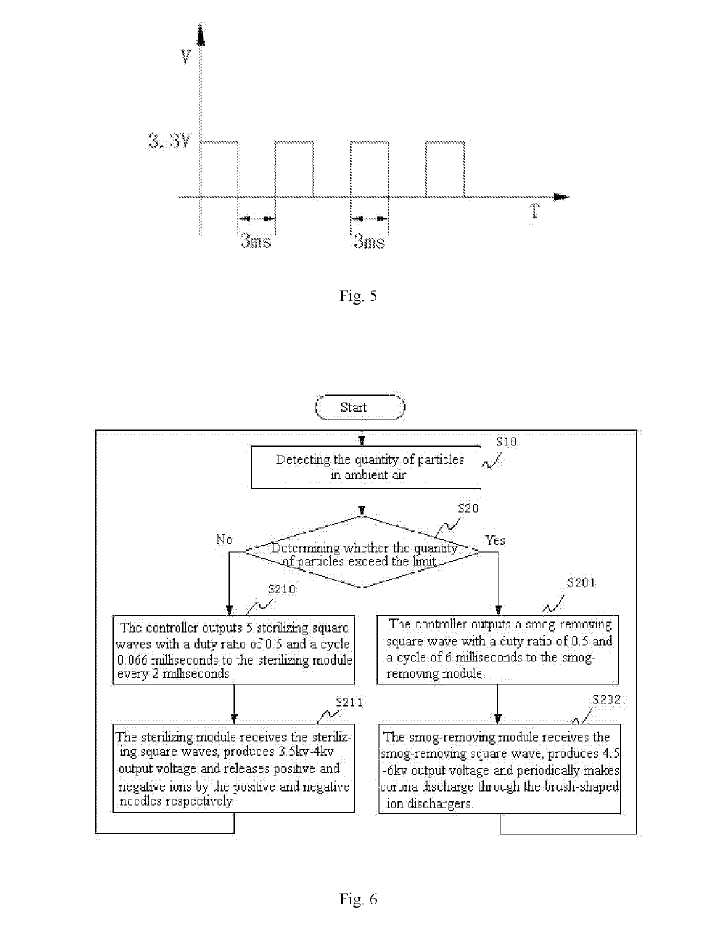

FIG. 5 is a control waveform from the controller of the ion-generating apparatus according to the present invention to the second triode.

As shown in FIG. 5, the controller U1 outputs a square wave with a duty ratio of 0.5 and a cycle of 6 milliseconds to the smog-removing module in order to achieve the purpose of corona discharge periodically. The cycle of six milliseconds is set up here for adapting to the circuit and the structure of the apparatus such that the apparatus can generate maximum negative ions almost without ozone, with the optimum smog-removing effect.

FIG. 6 is a flowchart of the method for sterilization and removing smog particles according to the present invention.

The foregoing description is made in details with reference to the examples and embodiment of the present invention, the exemplary embodiment has illustrated the principle and embodiment of the present invention. The embodiment described hereinbefore is for understanding the principle of the present invention and not for purposes of any restrictions or limitations on the invention. It will be apparent that any non-substantive, obvious alterations or improvement by the technician of this technical field according to the present invention may be incorporated into ambit of claims of the present invention.

1. An ion-generating apparatus for sterilization and removing smog, comprising:
a housing consisted of a shielding panel (1) and a casing (2), and
an ion generator configured within the housing for generating negative ions O₂⁻(H₂O)X and positive ions H⁺(H₂O)Y, wherein x and y are any natural numbers;
wherein the ion generator comprises a sterilizing module and a smog-removing module, both modules are dominated by one controller to generate positive and negative ions periodically.

2. The ion-generating apparatus of claim 1, wherein the sterilizing module comprises a sterilizing circuit consisting of a first transformation unit outputting 100 V-120 V voltage and a second transformation unit outputting 3.5 kV-4 kV voltage, a positive discharge needle (9A) and a negative discharge needle (9B); the controller, the first transformation unit and the second transformation unit are connected in turn.

3. The ion-generating apparatus of claim 2, wherein the first transformation unit further comprises a first transformer T1, a first diode D1 and a first resistor R1 connected in turn then connected to the first end of the primary coil of the first transformer, a first triode Q1 connected to the second end of the primary coil of the first transformer, and a second resistor R2 and a third resistor R3 connected in series one another then parallel-connected to the secondary coil of the first transformer; wherein the positive electrode of the first diode D1 is connected to the power source VCC and the negative electrode of the first diode D1 is connected to the first resistor R1, the emitter of the first triode Q1 is grounded and the base of the first triode Q1 is connected to the controller U1, and the connection end of the second resistor R2 and the third resistor R3 is connected to the controller U1.

4. The ion-generating apparatus of claim 3, wherein the second transformation unit further comprises a second transformer T2, a third diode S3 and a second diode S2 connected in series then connected to the first end of the primary coil of the second transformer T2, and a sixth diode D6 and a seventh diode D7 connected to the second end of the secondary coil of the second transformer T2, wherein the connection end of the third diode Q3 and the second diode Q2 is grounded through a first capacitor C1, the second end of the primary coil of the second transformer is grounded, the negative electrode of the sixth diode D6 is connected to the positive discharge needle (9A) and the positive electrode of the sixth diode D6 is connected to the second end of the secondary coil of the second transformer T2, and the positive electrode of the seventh diode D7 is connected to the negative discharge needle (9B) and the negative electrode of the seventh diode D7 is connected to the second end of the secondary coil of the second transformer T2.

5. The ion-generating apparatus of claim 2, wherein the smog-removing module comprises a smog-removing circuit and brush-shaped ion dischargers (4A, 4B), wherein the smog-removing circuit further comprises a third transformer T3, a fourth diode D4 and a sixth resistor R6 connected to the first end of the primary coil of the third transformer T3, a second triode Q2 connected to the second end of the primary coil of the third transformer T3, a fifth diode D7 and a seventh resistor R7 serial connected in turn and configured between the first end of the secondary coil of the third transformer T3 and the brush-shaped ion dischargers (4A, 4B), and a fourth resistor R4 and a fifth resistor R5 connected in series one another then parallel-connected with the secondary coil of the third transformer T3, wherein the positive electrode of the fourth diode D4 is connected to the power source VCC, the emitter of the second triode Q2 is grounded and the base of the second triode Q2 is connected to the controller U1, the connection end of the fourth resistor R4 and the fifth resistor R5 is connected to the controller, the positive electrode of the fifth diode D5 is connected to the seventh resistor R7 and the negative electrode of the fifth diode D5 is connected to the first end of the secondary coil of the third transformer T3, the output voltage of the secondary coil of the third transformer T3 is 4.5 kV-6 kV.

6. The ion-generating apparatus of claim 1, wherein the controller outputs five square waves with a duty cycle of 0.5 and a cycle of 0.066 milliseconds to the sterilizing module every two milliseconds.

7. The ion-generating apparatus of claim 1, wherein the controller outputs a square wave with a duty cycle of 0.5 and a cycle of 6 milliseconds to the smog-removing module.

8. The ion-generating apparatus of claim 5, wherein the shielding panel (1) comprises a first hole (7A) corresponding to the positive hydrogen ion discharge needle (9A), a second hole (7B) corresponding to the negative oxygen ion discharge needle (9B), and negative ions exhausting holes (8A, 8B) corresponding to the brush-shaped ion dischargers wherein there are two brush-shaped ion dischargers (4A, 4B) symmetrically provided inside the housing.

9. A method for sterilization and removing smog particles comprising the following steps:
detecting the quantity of particles in ambient air;
determining whether the quantity of particles exceed the limits; if yes, an ion generator produces negative and positive ions; otherwise, the ion generator periodically makes corona discharges which ionizes the air to form negative oxygen ions; then repeat the above steps.

10. The method of claim 9, wherein the ion generator as defined in any one of claims 1 to 8 produces negative and positive ions further comprising the following steps: a controller outputs five sterilizing square waves with a duty ratio of 0.5 and a cycle of 0.066 milliseconds to a sterilizing module every two milliseconds; the sterilizing module comprises a sterilizing circuit, a positive discharge needle and a negative discharge needle, the sterilizing circuit comprises a first transformation unit outputted 100 V-120 V voltage and a second transformation unit outputted 3.5 kV-4 kV voltage; the controller, the first transformation unit and the second transformation unit are connected in turn; and the sterilizing module receives the sterilizing square waves, produces 3.5 kV-4 kV output voltage, and releases positive and negative ions through positive and negative discharge needles respectively.

11. The method of claim 9, wherein the ion generator is as defined in claim 5 periodically makes corona discharge further comprising the following steps:
the controller outputs a smog-removing square wave with a duty ratio of 0.5 and a cycle of 6 milliseconds to a smog-removing module, and the smog-removing module which receives the smog-removing square wave, produces 4.5 kV-6 kV output voltage and periodically makes corona discharge through the brush-shaped ion dischargers (4A,4B).

12. Use of an apparatus as defined in any one of claims 1 to 8 for purifying air in an indoor environment.
